(19) United States
(12) Patent Application Publication
(10) Pub. No.: US 2023/0312887 A1
(43) Pub. Date: Oct. 5, 2023

(54) PROCESS FOR PREPARING SUGAMMADEX

(71) Applicants: Merck Sharp & Dohme LLC, Rahway, NJ (US); Werthenstein BioPharma GMBH, Schachen (CH)

(72) Inventors: David J. Lamberto, Jackson, NJ (US); Paolo Avalle, Luzern (CH); Lorenzo Codan, Olten (CH); Patrick Larpent, Lucerne (CH); Jochen Schoell, Luzern (CH); Jeffrey S. Neuhaus, Lansdale, PA (US)

(73) Assignees: Merck Sharp & Dohme LLC, Rahway, NJ (US); Werthenstein BioPharma GMBH, Schachen (CH)

(21) Appl. No.: 18/022,570

(22) PCT Filed: Sep. 8, 2021
(86) PCT No.: PCT/US21/49350

§ 371 Date: Feb. 22, 2023

Related U.S. Application Data
(60) Provisional application No. 63/076,133, filed on Sep. 9, 2020.

(51) Int. Cl. C08L 5/16; C08B 37/16

(52) U.S. Cl. CPC C08L 5/16; C08B 37/0012

(57) ABSTRACT

The present invention relates to a process for drying crystalline sugammadex to meet solvent specifications that is independent of API crystallinity or crystalline form generated. It further relates to use of sugammadex in the reversal of neuromuscular blockade induced by recuronium bromide or by vecuronium bromide in adults undergoing surgery.

### FIELD OF THE INVENTION

The present invention relates to a novel process for improving the preparation of the drug product BRIDION® (sugammadex).

### BACKGROUND OF THE INVENTION

Sugammadex is a modified cyclodextrin having the following structure:

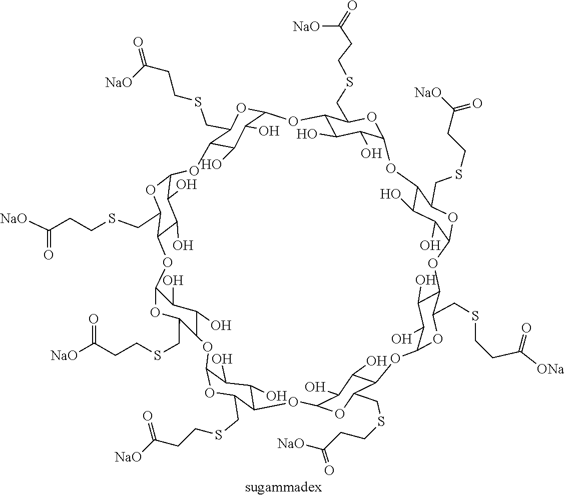

Sugammadex was approved in 2008 by the EMEA and in 2015 by the USFDA (and elsewhere) for the reversal of neuromuscular blockade induced by rocuronium bromide and vecuronium bromide in adults undergoing surgery. It is administered intravenously by injection in the form of a sterile solution under the brand name BRIDION®. Sugammadex is disclosed in WO2001/040316, published Jun. 7, 2001, together with a method for its synthesis. An improved synthesis of sugammadex is disclosed in PCT International Patent Application No. WO2019/236436, filed Jun. 3, 2019. Other methods of producing sugammadex are also disclosed in the art. Once produced, the active ingredient is typically isolated as a wet cake and then dried under vacuum to obtain a powder meeting purity and residual solvent specifications. The powder is then dissolved in water for injection, the pH adjusted, and the resulting solution is filtered and filled into vials, sterilized and stored for use. There remains a need in the art for an improved drying or purification process for sugammadex. The present invention addresses this need.

Sugammadex is a modified y-cyclodextrin active pharmaceutical ingredient (API) that is used as a reversal agent for neuromuscular blockade drugs in general anesthesia. The open structure of the cyclodextrin molecule yields a multitude of solid forms and to date more than 12 different mixed methanol solvate/hydrate forms have been characterized. Examples of crystalline forms of sugammadex are designated herein as crystalline form Type 1, crystalline form Type 2, crystalline form Type 3, crystalline form Type 8, and crystalline form Type 9, all disclosed in PCT application PCT/EP19/07582324604, filed Sep. 25, 2019, incorporated herein in its entirety. The crystalline forms are useful in the reversal of neuromuscular blockade induced by recuronium bromide and vecuronium bromide.

In the original process, the kinetic form (Type 1) was manufactured to ensure that the solids could be dried successfully using only heat and vacuum to meet desired specifications for residual solvents as described herein. Isolation of the thermodynamic and kinetic forms Type 2 and Type 3, respectively, were avoided due to the inability to remove process solvents to desired levels during drying and the subsequent need to rework the solids. Therefore, an improved drying process that is independent of crystallinity of the sugammadex starting material is desired to ensure robustness for meeting residual solvent levels of the final API at large scale. The instant invention relates to a mechanism for solvent removal independent of API crystallinity or crystalline form generated, wherein successful drying results in the displacement of solvent by water molecules, regardless of whether the crystal structure remains intact or collapsed.

Sugammadex is isolated as a crystalline solid that exists as several mixed methanol solvate/hydrate forms when isolated from methanol and water solvent systems. Water is a potent solvent for sugammadex; whereas methanol is added during the crystallization process as an antisolvent. Once dissolved in water and methanol, sugammadex tends to reach high levels of supersaturation before spontaneous nucleation occurs. Upon further addition of methanol, the kinetic Type 1 form readily nucleates. Type 2 and Type 3 were found to be more stable forms throughout the isolation process, featuring lower solubility compared to Type 1, Type 2 being the thermodynamic form. Type 8 and Type 9 were found to have comparable drying properties to Type 1, which does not require humid drying to remove the solvents. Nucleation of Type 2 without seeding or extended aging was rarely observed. In addition to solvent being more tightly bound in Type 2 and Type 3, the larger particle size of each is expected to be more difficult to dry based on a diffusion controlled drying mechanism where the drying rate can be slowed by the square of the thickness of the particles. FIG. 1 shows scanning electron micrographs of the Type 1, Type 2, and Type 3 forms: Type 1 exists primarily as agglomerated platelets whereas Type 2 and Type 3 feature faster growth kinetics and tend to form larger crystals. Type 2 morphology has been observed to vary between blocks, rods, and plates or a mix of each whereas Type 3 particles can have a block-like or rod-like appearance.

For the initial manufacturing process, Type 1 platelets were selected as the target forms due to the ease of formation and the ability to remove the process solvents to meet specifications set as <200 ppm methanol, <5 wt % ethanol, and <10 wt % water. Once formed, the solids were filtered and then washed with a mixture of methanol-denatured ethanol and water. Though the exact stoichiometry of the solvents and water was not known, it was believed that ethanol from the wash solution can partially substitute for methanol in the crystal lattice. Therefore, washing with an ethanol solution reduced the methanol content on the solids entering the dryer and thereby reduced the burden of methanol removal during the subsequent drying unit operation.

The Type 1 solids were then readily dried to specification using only heat and vacuum. However, production batches have sometimes failed due to elevated residual solvent content, requiring rework of the solids. Investigations concluded that batch failures were due to undesired seeding by residual solids within the process train and subsequent formation of Type 2 and/or Type 3 crystals. The inability to meet residual solvent specifications was caused by both crystalline form and larger particle size. As a result, strict inter-batch cleaning protocols were implemented to effectively eliminate any potential seed source. Additionally, hold times were limited post methanol addition to avoid direct formation of Type 2 or Type 3 or turnover from Type 1 to Type 2 or Type 3 upon aging.

Ultimately, a recent increase in demand for sugammadex necessitated additional process improvements to meet projected volumes. Therefore, process development was initiated to generate the thermodynamically preferred Type 2 form including an improved drying procedure for consistently meeting residual solvent specifications for this form. Previous studies had shown for other compounds, that maintenance of the crystalline hydrated form, either through use of humidified drying gas or careful control of the dying conditions, was critical for the successful removal of process solvents (Lamberto, D. J., et al., Org Process Res Dev 2017, 21, 1828-1834; Khoo, J. Y. et al., Ind Eng Chem Res 2010, 49, 422-427; Adamson, J. et. al., Org Process Res Dev 2016, 20, 51-58. However, in the case of sugammadex, maintenance of the crystalline form was not possible (or desirable) since the form consists of both a solvate and a hydrate. Even if water was maintained in the crystalline lattice, methanol was necessarily removed during drying and the starting form was lost. The present invention provides an approach to successful drying by solvent displacement using water in the drying gas to accelerate the removal of solvents and enable lower levels of solvent to be achieved in the final dry sugammadex solids without concern of the crystal type. There is need for a process that is capable of drying all forms of sugammadex solids, including crystalline form Type 2 and crystalline form Type 3 to desired levels of impurities, particularly residual solvents and water, as this was previously not possible using dry nitrogen flow or applying combinations of heat and vacuum.

### SUMMARY OF THE INVENTION

The present invention relates to a process for improving the preparation of drug product BRIDION (sterile solution). More particularly, the present invention relates to a novel process for making the pharmaceutical product sugammadex through the use of an improved drying process for sugammadex. The present invention further relates to a process for drying crystalline sugammadex to meet solvent specifications that are independent of API crystallinity or crystalline form generated.

In one aspect, there is provided a process for drying crystalline sugammadex wet cake:

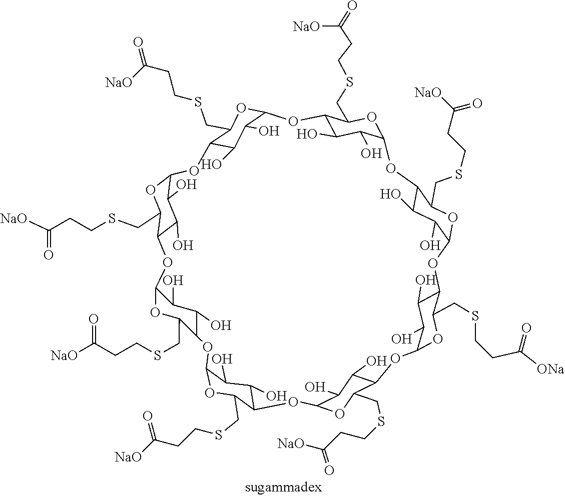

comprising
1) flowing humid gas through a wet cake of crystalline sugammadex, water, and solvent, said gas having a relative humidity of about 15% to about 75%,
2) isolating the sugammadex product.

The present invention further relates to novel crystalline forms of sugammadex, designated herein as crystalline form Type 5 of sugammadex and crystalline form Type 11 of sugammadex, pharmaceutical compositions thereof, and methods of use in the reversal of neuromuscular blockade induced by recuronium bromide and vecuronium bromide in adults undergoing surgery.

In one aspect, the present invention provides novel crystalline forms of sugammadex. In one embodiment, there is provided crystalline form Type 5 of sugammadex. In another embodiment, there is provided crystalline form Type 11 of sugammadex.

In another aspect, the present invention provides methods for the use of each of the aforementioned crystalline forms of sugammadex in the preparation of a medicament for use in the reversal of neuromuscular blockade induced by rocuronium bromide and vecuronium bromide in adults undergoing surgery in accordance with its approved label.

The examples provided herein are for illustrative purposes so that the invention may be more fully understood. These examples should not be construed as limiting the invention in any way.

### BRIEF DESCRIPTION OF THE DRAWINGS

FIG. 1A-1C Scanning electron micrographs with a magnification of 250× of (1A) Type 1 agglomerated platelets; (1B) Type 2 mix of blocks and large plates; and (1C) Type 3 blocky and rod-like plates.

FIG. 2A-2B Comparison of drying profiles when using (2A) humid and (2B) dry nitrogen flow. Ethanol, methanol, and water were monitored in the drying gas using mass spectrometry. Raman spectroscopy was used to track the conversion to the final dry form.

FIG. 3 Schematic depicting impact on drying conditions and drying time on residual solvent levels.

FIG. 4 is a graph of a Powder X-Ray Diffraction (“PXRD”) pattern of sugammadex Type 1 crystalline form, generated using the equipment and methods described herein. The graph plots the intensity of the peaks as defined by counts per second versus the diffraction angle 2 theta (2θ) in degrees.

FIG. 5 is a graph of a Powder X-Ray Diffraction (“PXRD”) pattern of sugammadex Type 2 crystalline form, generated using the equipment and methods described herein. The graph plots the intensity of the peaks as defined by counts per second versus the diffraction angle 2 theta (2θ) in degrees.

FIG. 6 is a graph of a Powder X-Ray Diffraction (“PXRD”) pattern of sugammadex Type 3 crystalline form, generated using the equipment and methods described herein. The graph plots the intensity of the peaks as defined by counts per second versus the diffraction angle 2 theta (2θ) in degrees.

FIG. 7 Drying profile collected during DOE Run #8.

FIG. 8 Drying profile of pilot plant batch (25 kg).

FIG. 9 is a graph of a Powder X-Ray Diffraction (“PXRD”) pattern of sugammadex Type 5 crystalline form, generated using the equipment and methods described herein. The graph plots the intensity of the peaks as defined by counts per second versus the diffraction angle 2 theta (2θ) in degrees.

FIG. 10 is a graph of a Powder X-Ray Diffraction (“PXRD”) pattern of sugammadex Type 11 crystalline form, generated using the equipment and methods described herein. The graph plots the intensity of the peaks as defined by counts per second versus the diffraction angle 2 theta (2θ) in degrees.

FIG. 11 is a graph of a Powder X-Ray Diffraction (“PXRD”) pattern of sugammadex Type 13 crystalline form, generated using the equipment and methods described herein. The graph plots the intensity of the peaks as defined by counts per second versus the diffraction angle 2 theta (2θ) in degrees.

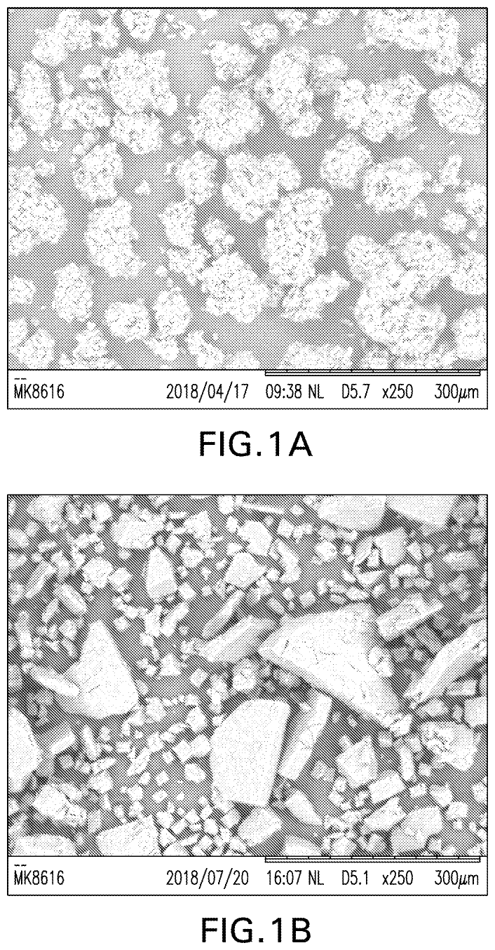

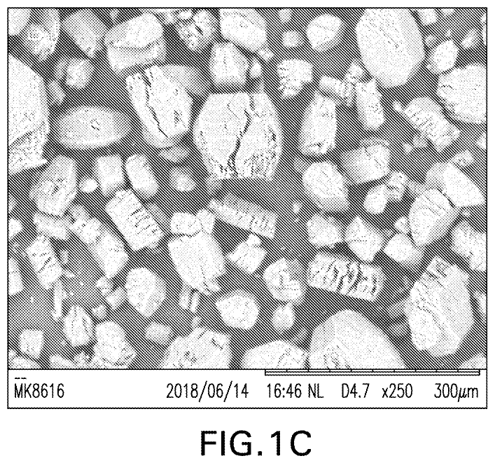

### DETAILED DESCRIPTION OF THE INVENTION

### Abbreviations and Definitions

The terms used herein have their ordinary meaning and the meaning of such terms is independent at each occurrence thereof. That notwithstanding and except where stated otherwise, the following definitions apply throughout the specification and claims.
API: active pharmaceutical ingredient
° C. means degrees Celsius
DOE: design of experiment
EtOH: ethanol
FIG (or FIG. or Fig. or Fig or fig. or fig) means Figure (or figure) and refers to the corresponding figure
g means gram (or grams)
H (or h): hour(s)
HPLC: high pressure liquid chromatography
KF: Karl Fischer titration
MeOH: methanol
min: minute(s)
mL means milliliter (or milliliters)
mmHg: millimeters of mercury
PXRD: powder x-ray diffraction
ppm: parts per million
r.t. (or R.T.): Room Temperature
V or v: volume(s) is defined as the amount of solvent used based on the amount of the relevant limiting reagent; i.e., 1V (or 1v)=1 ml solvent for each gram of the limiting reagent.
w %=weight percent
v/v or (v:v:v) refers to a mixture of liquids by volume

Solvents and reagents that are commercially available were used as received. All solvents and reagents indicated as being commercially available may be obtained from many commercial suppliers, including, e.g., Sigma Aldrich, St. Louis, MO, USA.

### PREPARATION OF CRYSTALLINE SUGAMMADEX

In one aspect, this invention relates a process for drying crystalline sugammadex wet cake:

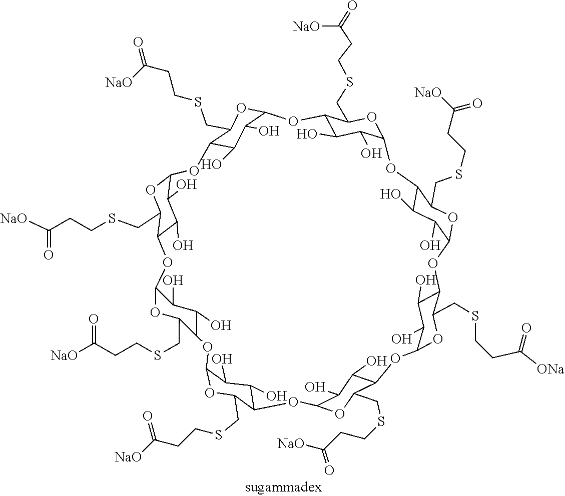

comprising
1) flowing humid gas through a wet cake of sugammadex, water, and solvent, said gas having a relative humidity of about 15% to about 75%,
2) isolating the sugammadex product.

In another aspect of this invention, the gas is selected from: argon, helium, nitrogen, and oxygen. Another subembodiment of this aspect of the invention is realized when the gas is nitrogen.

In another aspect of this invention, the solvent is selected from ethanol or methanol. In another aspect of this invention, the solvent is selected from a mixture of ethanol and methanol.

In an embodiment of this invention the solvent is substantially methanol. In another embodiment of this invention the solvent is substantially ethanol. Still in another embodiment of this invention the solvent is a mixture of methanol and enthanol.

In another aspect of this invention, the relative humidity of the nitrogen is about 25% to about 60% and the temperature is maintained from about 25° C. to about 50° C., preferably the temperature is maintained from about 25° C. to about 35° C. A subembodiment of this aspect of the invention is realized when the relative humidity of the nitrogen is about 25% to about 60% and the temperature is maintained from about 25° C. to about 35° C.

In another aspect of the invention, the temperature is maintained from about 25° C. to about 50° C. to remove residual solvent.

In another aspect of this invention, the humid gas is nitrogen with a relative humidity of about 40% to about 45% and the temperature is maintained at about 25° C. to about 50° C., preferably from about 25° C. to about 30° C.

In another aspect of the invention, before isolating the sugammadex product, residual water level is optionally reduced by applying a dry gas flow or vacuum. An embodiment of this aspect of the invention is realized when the dry gas is selected from argon, helium, nitrogen, and oxygen. An embodiment of this aspect of the invention is realized when the dry gas is nitrogen. In another aspect of this invention, residual water is reduced using a dry nitrogen flow. In another aspect of this invention, residual water is reduced using vacuum. In another aspect of this invention, residual water is optionally reduced using a dry nitrogen flow or vacuum at a temperature from about 25° C. to about 50° C.

In another aspect of the invention, residual water level is optionally reduced by adjusting the drying temperature while maintaining the relative humidity at the level used in the humid stage. In yet another aspect of the invention, residual water level is optionally reduced by reducing the relative humidity while maintaining a constant drying temperature.

Still in another aspect of this invention, pressure during the humid drying process is maintained from 25 mmHg to 475 mmHg, preferably from 250 mmHg to 350 mmHG, more preferably 250 mmHg.

In another aspect of the invention, the process provides sugammadex with a residual water level of less than or equal to 20 wt %. In another aspect of the invention, the process provides sugammadex with a residual water level of less than or equal to 10 wt %. In another aspect of the invention, the process provides sugammadex with residual methanol level less than 1000 ppm. In another aspect of the invention, the process provides sugammadex with residual methanol level of less than or equal to about 500 ppm. In another aspect of the invention, the process provides sugammadex with residual methanol level of less than or equal to about 200 ppm. In another aspect of the invention, the process provides sugammadex with residual ethanol level of less than or equal to about 5 wt %. In another aspect of the invention, the process provides crystalline sugammadex with residual water, methanol and ethanol levels of less than or equal to about 20 wt % water, less than or equal to about 1000 ppm methanol, and less than or equal to about 5 wt % ethanol. In another aspect of the invention, the process provides crystalline sugammadex with residual water, methanol and ethanol levels of less than or equal to about 10 wt % water, less than or equal to about 500 ppm methanol, and less than or equal to about 5 wt % ethanol. Still in another aspect of this invention, the process provides crystalline sugammadex with residual water, methanol and ethanol levels of less than or equal to about 10 wt % water, less than or equal to about 200 ppm methanol, and less than or equal to about 5 wt % ethanol.

Another embodiment of this aspect of the invention is realized when the gas flow is directed down through the solids and does not involve a sweep.

Sugammadex made using the humid drying process described herein may be prepared according to the procedures described below. For each procedure, starting quantities of sugammadex wet cake of different crystal forms e.g., Type 1, Type 2, Type 3, Type 8, Type 9, etc., may be obtained from any suitable synthesis, including those described in PCT Publication No. WO2001/040316, Zhang, et al., published Jun. 7, 2001; and WO2019/236436, filed Jun. 3, 2019 and PCT Application PCT/EP2019/075823, Attorney Docket 24604, file Sep. 25, 2019, all incorporated herein by reference, and those described below.

Humid drying experiments were conducted using drying flow cells consisting of jacketed glass vessels as described by Lamberto, et al., Org Process Res Dev 2017, 21, 1828-1834.2017. During the experiments, the drying gas was introduced at the top of the cell and flowed through the solids sitting on a frit located at the bottom of the cell to ensure reproducible gas-solid contact. The system temperature and pressure, and the flow rate and humidity of the inlet gas stream were controlled independently to desired set points. Agitation during the runs was not necessary as length scales for heat and mass transfer were small in the experimental setup (temperature changes of the jacket fluid were observed by a response at the center of the cake within seconds). Drying gas flow rate was reduced to scale with the dryer area or product mass at large scale. Values of all drying parameters were recorded continuously throughout each experiment. Process analytical technology (PAT) was used to monitor the volatile components in the exiting gas stream using mass spectrometry while the crystalline form of the drying solids was tracked using Raman spectroscopy. Alternatively, the Raman probe could be replaced with a thermocouple to monitor the temperature of the cake during drying.

### Process Analytical Technology (PAT) and Offline Methods Used

The PAT tools used to support the work performed during this study were Mass Spectrometry (MS) using a Proline Dycor system (Ametek, Pittsburgh, PA), Raman Spectroscopy using a fiber optic probe connected to an RamanRxn2 Analyzer (Kaiser Optical Systems, Inc., Ann Arbor, MI), and humidity and temperature measurement using a HMP60 humidity and temperature sensors from Vaisala (Helsinki, Finland).

Solvent content of the solids was determined using a headspace gas chromatograph equipped with a J&W DB-624 column (Agilent, Santa Clara, CA) and water content was determined using a Karl Fischer Coulometer with oven (Metrohm, Switzerland).

### PXRD

To determine the level of crystallinity and form, powder x-ray diffraction (PXRD) measurements were carried out on a Bruker D8 Advance System configured in the Bragg-Brentano configuration and equipped with a Cu radiation source with monochromatization to Kα achieved using a nickel filter. A fixed slit optical configuration was employed for data acquisition. Data were acquired between 3 and 40° 2θ and a step size of 0.018. Samples were prepared by gently pressing the samples onto a shallow cavity zero background silicon holder. Wet cake samples were covered with Kapton® (polyimide film, DuPont, USA) foil in order to maintain the wet-sample-condition throughout data collection.

Those skilled in the art will recognize that the measurements of the PXRD peak locations for a given crystalline form of the same compound will vary within a margin of error. The margin of error for the 2-theta values measured as described herein is typically +/−0.2° 2θ. Variability can depend on such factors as the system, methodology, sample, and conditions used for measurement. As will also be appreciated by the skilled crystallographer, the intensities of the various peaks reported in the figures herein may vary due to a number of factors such as orientation effects of crystals in the x-ray beam, the purity of the material being analyzed, and/or the degree of crystallinity of the sample. The skilled crystallographer also will appreciate that measurements using a different wavelength will result in different shifts according to the Bragg-Brentano equation. Such further PXRD patterns generated by use of alternative wavelengths are considered to be alternative representations of the PXRD patterns of the crystalline material of the present invention and as such are within the scope of the present invention.

Initial Laboratory Sugammadex Drying Runs

To assess the drying performance of these solids, a series of experiments were conducted to evaluate the effect of various washing and drying protocols to enable drying to desired specification. These experiments included combinations of both drying with and without humidity and the use of a standard and an aged wash procedure. Unless otherwise noted, the standard washing procedure consisted of two, 3V, displacement washes using standard ethanol wash (86:4:10 vol % of ethanol:methanol:water) while the age wash involved soaking the solids in a larger quantity of the standard ethanol wash for an extended amount of time. The upper end of the wash volumes was set to 8 volumes based on the estimated capacity of the filter dryer at scale.

The experiments in Table I were conducted on a 10 g scale using Type 2 wet solids with approximately 11 wt % methanol, 25 wt % ethanol and 12 wt % water. Each experiment was conducted for a fixed duration of ˜17 hours while the gas flow rate was held constant at 100 mL/min standard flow (sccm). The desired specifications after humid drying, with or without an additional step of non-humid drying, for the sugammadex dry cake at the end of the process are (a) residual water levels are less than or equal to 20 wt %, or less than or equal 15%, or less than or equal to 10%, (b) residual methanol levels are less than 1000 ppm, or less than 500 ppm, or less than or equal to 200 ppm, and (c) residual ethanol levels are less than or about 5 wt % ethanol.

Thus, an aspect of the invention is realized when the sugammadex dry cake at the end of the humid drying process meets specification which is residual water, methanol and ethanol levels of less than or equal to 15 wt %, less than or equal to about 500 ppm, and less than or equal to about 5 wt %, respectively. Another aspect of the invention is realized when a lower limit specification for the sugammadex dry cake at the end of the humid drying process is residual water of less than or equal to 10 wt %, methanol less than or equal to about 200 ppm and ethanolless than or equal to about 5 wt %. The operating conditions and residual solvent levels achieved in these drying experiments were summarized in Table I.

TABLE I
Summary of initial Sugammadex Drying Runs

| | Inlet | Press | Temp | EtOH | MeOH | Water |
|---|---|---|---|---|---|---|
| Drying Run # (description) | RH % | (mmHg) | (° C.) | [wt. %] | [ppm] | [wt %] |
| 1 (dry N₂ low pressure) | <1 | 25 | 40 | 7.4* | 2147* | 1.5 |
| 2 (dry N₂ low pressure) | <1 | 25 | 40 | 7.6* | 2858* | 1.8 |
| 3 (dry high press./low temp.) | <1 | 250 | 22 | 6.9* | 2035* | 1.6 |
| 4 (vacuum only) | na | ≤25 | 18 | 7.7* | 10720* | 11.3* |
| 5a (humid low pressure) | 45 | 25 | 40 | 4.2 | 232 | 5.7 |
| 5b (humid low pressure) | 55 | 25 | 40 | 3.9 | 88 | 6.2 |
| 6 (humid high pressure) | 60 | 350-250 | 40 | 1.8 | 62 | 7.4 |
| 7 (humid high pressure) | 60 | 250 | 40 | 1.8 | 52 | 7.5 |
| specifications | | | | <5 | <200 | <10 |

Runs 1 through 4 successfully replicated the poor drying behavior (designated by *) observed at larger scale for crystal form Type 2 solids. In the first two experiments, dry nitrogen, defined as having a relative humidity less than 1%, was passed through the solids while the system outlet pressure and jacket temperature were controlled to 25 mmHg absolute and 40° C. respectively. As can be seen in the first and second rows of Table I, under these conditions, incomplete removal of the residual process solvents was observed with residual levels of ethanol and methanol averaging 7.5 wt % and 2503 ppm respectively. Similar results were obtained when operating at higher pressure and lower temperature as shown in row 3. All three experiments resulted in failing solvent levels while water content in the solids was relatively low. Drying conducted using vacuum only without nitrogen flow was found to be ineffective for removal of both solvents and water (row 4).

Conversely, the presence of water in the drying gas enabled more complete removal of the process solvents. In experiment 5a, the wet solids were dried under identical conditions used in experiment 1 except that the humidity of the nitrogen stream was increased to 45%. The increased humidity resulted in lower residual solvent levels with ethanol passing at 4.2 wt % and methanol at 232 ppm. Continued drying of these solids at an increased humidity of 55% yielded improved methanol levels of 88 ppm (experiment 5b).

Successful drying of the Type 2 form of sugammadex at higher pressure was also demonstrated and documented in experiments 6 and 7 in Table I. In these runs, the humidified nitrogen gas was passed through the solids at a flow rate of 100 sccm while the system outlet pressure was controlled at 250 mmHg, and the temperature of the drying vessel jacket was maintained at 40° C. (Note, higher pressure was used during the initial 70 minutes of experiment 6 before it was reduced from 350 mmHg to 250 mmHg). In both cases, the residual solvents and water levels were all well within desired specification. Operating at increased pressure (250 versus 25 mmHg) did not hinder solvent removal. and in fact the residual solvent levels were lower than those obtained from experiments run at lower pressure for the same amount of time.

Humidity Post Initial Drying with Dry Nitrogen

The experiments in Table II using Type 2 solids were first dried for 17 hours using dry nitrogen flow (1a) and then subsequently rehydrated by repeated 17-hour exposures to humidified nitrogen streams (1b and 1c).

TABLE II
Humidity post initial drying with dry nitrogen

| | Inlet | Press | Temp | EtOH | MeOH | Water | |
|---|---|---|---|---|---|---|---|
| Drying Run # (description) | RH % | (mmHg) | (C.) | [wt. %] | [ppm] | [wt %] | Crystallinity |
| 1a (dry N₂ low pressure) | <1 | 25 | 40 | 7.6* | 2858* | 1.8 | low |
| 1b (humid low pressure) | 55 | 25 | 40 | 5.6* | 169 | 5.7 | low |
| 1c (humid low pressure) | 55 | 25 | 40 | 5.3 | 71 | 5.5 | low |
| specifications | | | | <5 | <200 | <10 | not specified |

The results shown in the first row of Table II indicate that the use of dry nitrogen flow generated solids with residual solvent levels (as represented by *) that do not meet specification and had relatively low water content. However, further reduction of the residual solvent levels as possible with a subsequent exposure to a nitrogen stream with a higher relative humidity (second row). The reduction in solvent content continued with repeated exposure to the humidified stream (third row). Although this solvent removal was accompanied by an increase in residual water levels, the level of crystallinity and form, as determined by PXRD, remained unchanged as indicated in the last column of Table II. This suggests that solvent removal was not controlled by maintaining or regaining crystallinity as reported for other cases in the literature, but rather by displacement of the solvent by water in the drying gas. However, it should be noted that high residual water alone was not sufficient for solvent removal as was discussed in the previous section regarding “vacuum only” drying in Table I. Hence, it can be concluded that additional water entering with the drying gas was critical for achieving lower residual solvent levels.

Drying Runs Using Humid and Dry Nitrogen Flow

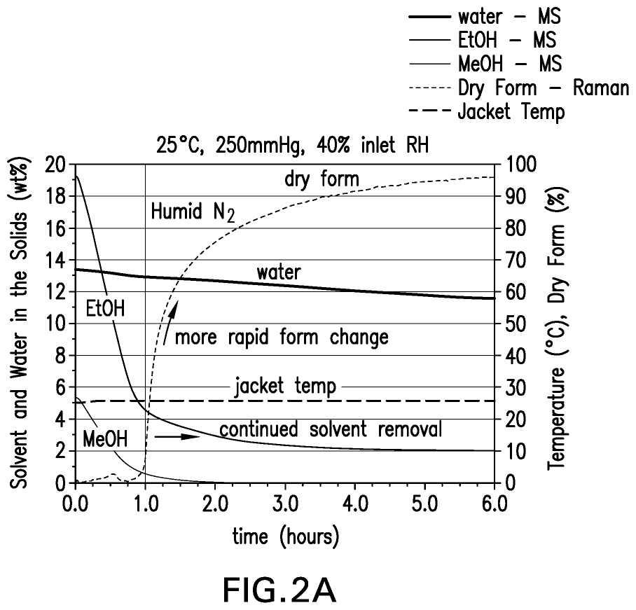

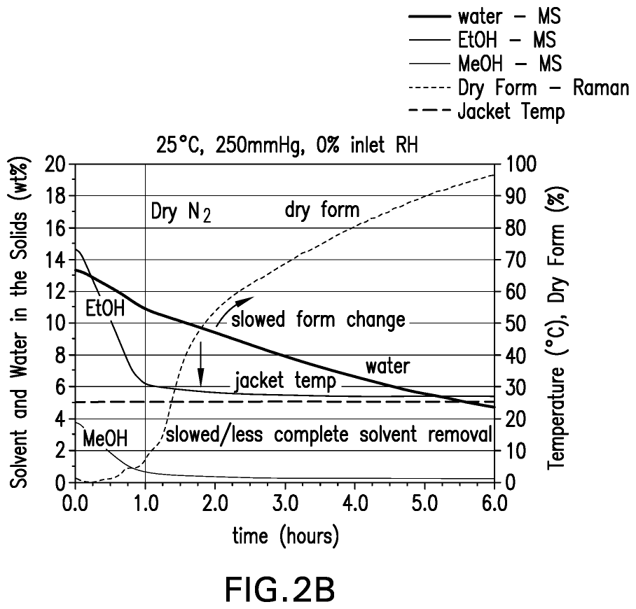

Residual solvents and water content for drying runs using humid and dry nitrogen flow conditions were also observed in further experiments in which two runs were conducted under identical conditions except for the humidity of the drying gas. The drying experiments were performed at 25° C. and 250 mmHg but one used nitrogen with a relative humidity of 40% while the other used dry nitrogen with a relative humidity of <1%. In both experiments, the solvents and water coming off the solids were monitored by mass spectrometry and form transition was tracked by Raman spectroscopy. FIG. 2 shows a comparison of solvent and water contents in the solids as a function of drying time as determined using the starting and ending solvent and water content and integrating the respective mass spectrometry signals for the two runs.

It can be readily observed that the behavior during both experiments resembled classical constant rate/falling rate drying kinetics: The time to reach the end of the constant rate period was similar in both runs and occurred just prior to the 1-hour point of drying as shown by the methanol and ethanol curves. However, drying with humid nitrogen flow shown in FIG. 2 (a) was not only more efficient as revealed by the steeper slope of the methanol and ethanol curves, but solvent removal also continued to a much greater extent over an extended time. Moreover, form change occurred more quickly when humid nitrogen was employed during the drying process. Contrarily, form conversion proceeded more slowly, and solvent was removed to a lesser extent for the case of dry nitrogen flow shown on the right. The transition from the constant rate to the falling rate regimes occurs at a lower solvent with humid drying despite higher starting solvent content, suggesting that water was penetrating the solids and was more readily displacing solvent from the crystalline lattice earlier in the drying process.

Looking at the end point results of the solids listed in the Table III, water in the drying gas not only accelerated solvent removal but also resulted in more complete removal and lower levels overall as compared with use of dry nitrogen flow.

TABLE III
Residual solvents and water content for drying
runs using humid and dry nitrogen flow

| Drying Run | EtOH [wt. %] | MeOH [ppm] | Water [wt. %] |
|---|---|---|---|
| Humid N₂ | 1.8 | 70 | 10 |
| Dry N₂ | 5.3* | 2630* | 2 |
| Specifications | <5 | <200 | <10 |
| (lower limits) | | | |

Although form conversion was occurring as solvents were removed, this did not hinder continued solvent removal when humid nitrogen was used. No difference in the level of crystallinity was observed for the solids obtained from each run. These results further support that displacement of the solvent by water was driving solvent removal and that maintaining crystallinity was not a critical mechanism for the drying process. It should be noted that, following solvent removal using humid drying, reduction of residual water content to below 10 wt % was easily achieved using dry nitrogen flow (data not shown).

### Drying Temperature, Pressure and Humidity

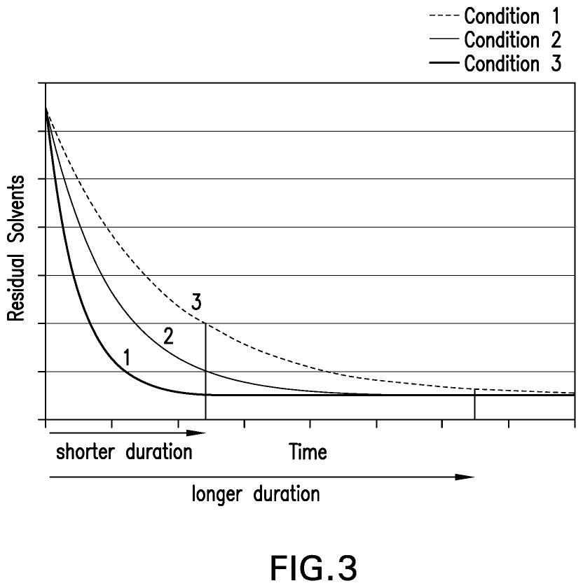

In additional studies, drying temperature, pressure, and the humidity of the drying gas were further investigated. A relatively simple but effective three factor, full factorial design of experiments (DOE) with 2 center point replicates was executed with the primary responses measured being residual ethanol, methanol, and water content of the solids. Secondary responses such as the cake temperature, pressure drop across the solids, purity, crystallinity, and form of the dried solids were also monitored. Additional factors of drying gas flow rate and drying time were fixed for the DOE and the impact of each was considered separately in later experiments. The drying time was kept relatively short for all runs to emphasize differences in drying efficacy due to the processing conditions as depicted in the schematic in FIG. 3.

For jacket temperature, the low and high values were set to 20 and 50° C. respectively, with a center point of 35° C., while pressure values were set to 25 and 475 mmHg, with a center point of 250 mmHg. The inlet relative humidity low and high targets were 40% to 80% respectively, with a center point of 60%. The high end of the humidity was then adjusted down to 75% after initial testing indicated that it might not be possible to achieve 80% for all runs at the laboratory scale. The drying gas flow rate and the mass of wet solids charged were fixed at 100 sccm and ˜8 g respectively (˜1.5 cm of cake in the 25 mm ID flow cell). Finally, the drying time was fixed at 7 hours. This shorter time was selected to emphasize the differences in run conditions and highlight the impact of the DOE factors. Residual solvent levels would be expected to be reduced in some instances with extended drying times.

A common stock of wet solids was prepared by conducting several Type 2 crystallization batches, filtering the solids, and washing with the standard wash solution consisting of a mixture of methanol-denatured ethanol and water (86:4:10 vol % ethanol, methanol, water). The solids from these batches were consolidated and used for the DOE runs. Samples of the wet cake were taken before each run and checked for solvent content to ensure the starting point for all runs was consistent (starting solvent content was ˜25 wt % EtOH, ˜8 wt % MeOH, and ˜13 wt % water).

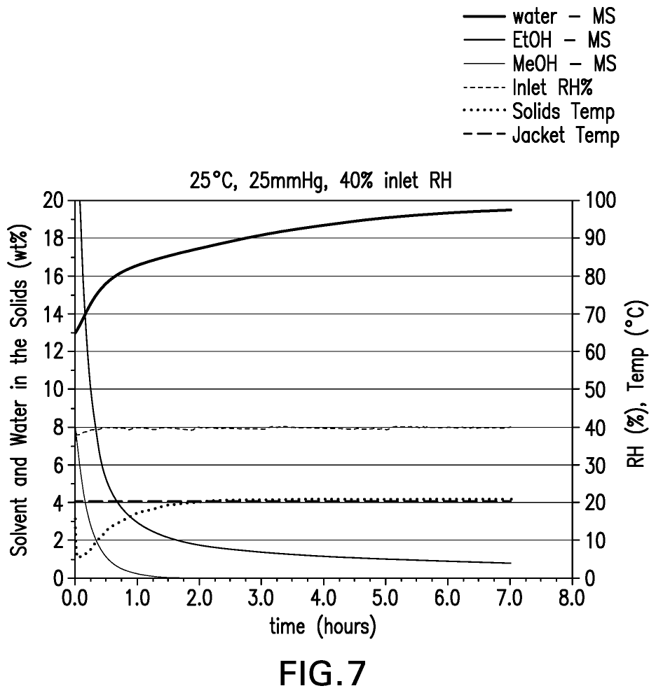

The order of the experiments was randomized, and the DOE was executed. Conditions were monitored in real time with PAT data collected throughout. The full spectrum of data generated from online PAT is shown in FIG. 7 which depicts the drying profile generated during DOE run 8. In this experiment, the jacket temperature of the drying flow cell was set to 20° C., the outlet pressure was controlled at 25 mmHg, and the humidity of the inlet drying gas was adjusted to 40%. During the DOE runs, a thermocouple was installed in place on the Raman probe to monitor the change in the temperature of the solids over the course of drying.

Solvent removal was again monitored by mass spectrometry, and the solvent content and water content in the solids were determined by integration. Like all runs, removal of unbound (i.e., physically adsorbed) solvent was very rapid at the start of drying and slowed as only lattice bound solvent remained. The change in cake temperature (FIG. 7) was consistent with this drying behavior and quickly dropped to a minimum of ˜5° C., due to evaporative cooling, during the constant rate period and then increased more slowly to the jacket temperature (FIG. 7) as solvent removal rate decreased over time. The mass spectrometry data suggests that the transition to the falling rate period for this run started after about 30 minutes of drying and became fully mass transfer limited at the 2-hour point when the cake temperature was equal to the jacket temperature.

In FIG. 7 it can be readily observed in the slope of water signal that the uptake of water was faster at the start of drying as compared to the end. This suggests that a larger portion of incoming water was being absorbed by the solids at a point in the drying process when the solvent removal was high and solid temperature was low. The water content of the solids continued to increase as equilibrium with the gas phase was approached and solvents were removed to very low levels (0.8 wt % ethanol and 50 ppm MeOH). The slight increase in the temperature of the solids above the jacket temperature was attributed to this water absorbance in the absence of evaporative cooling.

The residual solvent and water results from this and the other DOE runs were summarized along with the drying conditions and specifications in Table IV. Results not within desired specification are designated with an *.

TABLE IV
Summary of DOE Results

| | Temp | Press | Inlet | EtOH | MeOH | Water | |
|---|---|---|---|---|---|---|---|
| DOE# | (C.) | (mmHg) | RH % | [wt. %] | [ppm] | [wt %] | Crystallinity |
| 1 | 35 | 250 | 60 | 2.7 | 230 | 9.8 | Medium |
| 2 | 50 | 25 | 40 | 6.3* | 1930* | 3.8 | Low |
| 3 | 20 | 475 | 75 | 1.6 | 380 | 21.8 | Medium |
| 4 | 50 | 475 | 75 | 2.1 | 140 | 6.0 | Medium |
| 5 | 20 | 25 | 75 | 0.5 | 130 | 27.2 | High** |
| 6 | 50 | 475 | 40 | 2.7 | 310 | 4.7 | Low |
| 7 | 50 | 25 | 75 | 3.6 | 280 | 5.6 | Medium |
| 8 | 20 | 25 | 40 | 0.8 | 50 | 19.5 | Low |
| 9 | 20 | 475 | 40 | 2.0 | 150 | 18.5 | Low |
| 10 | 35 | 250 | 60 | 2.1 | 140 | 10.2 | Low |

| | | | | |
|---|---|---|---|---|
| Specifications (lower limits) | <5 | <200 | <10 | not specified |

**The form of dry solids from all runs was identical except for run#5 which was a new hydrate

Some initial trends in the data in Table IV were clear prior the use of statistical analysis. As can be seen in Table IV, the removal of ethanol to below the specification of less than 5 wt % was not problematic with only one set of conditions (DOE #2) leading to result that did not meet specification. Methanol and water showed good sensitivity to the selected drying conditions with a mix of both passing and failing results obtained. The data show that the residual water levels were lower for cases where the temperature was higher. In addition, conditions resulting in lower water levels lead to higher residual methanol content (runs 6 and 7 versus 8 and 9) and in the extreme case (run 2), where temperature was high, and pressure and humidity were low, both methanol and ethanol were failing. The relationship between residual water and solvent levels further supports a diffusion controlled drying mechanism where solvent removal is driven by displacement by water.

The level of crystallinity again did not correlate with the residual solvent content. All solids were consistent as the typical dry form except for the solids from run 5, which were confirmed to be a new hydrate (Type 13), as measured by powder x-ray diffraction, PXRD). Although higher water content typically led to lower residual solvent levels, care was needed to avoid conditions leading to deliquescence and the formation of gooey solids as was observed during runs 3 and 5. This was not unexpected as these runs were both conducted at conditions of low temperature and high humidity. The center points (runs 1 and 10) yielded free flowing powder while others (runs 2, 4, 6, and 7) yielded aggregated solids which broke up into free-flowing powders through mixing using a spatula. The solids generated during runs 8 and 9 had similar water content (19-20 wt %) and consisted of loose powders that exhibited some increased resistance to flow when mixed.

Additional experiments were completed where Type 1, 2, and 3 solids were washed with a solution of 8:1 MeOH:water (free of any ethanol) prior to drying under dry and humid conditions. The solvent and water content of the initial and final solids as well as the drying conditions are provided in the Table V below.

TABLE V

| | | Temp | Press | Inlet | NMP | MeOH | Water |
|---|---|---|---|---|---|---|---|
| | Sample | (C.) | (mmHg) | RH % | [ppm] | [ppm] | [wt %] |
| Type | Wet Cake | n/a | n/a | n/a | 210 | 447000 | 11 |
| 1 → 2 | Dry Cake - Dry N2 | 50 | 50 | 0 | 90 | 36300* | 1.1 |
| | Wet Cake | n/a | n/a | n/a | 45 | 293000 | 10.5 |
| | Dry Cake - Humid | 35 | 250 | 60 | 44 | 107 | 11.7 |
| Type 2 | Wet Cake | n/a | n/a | n/a | 188 | 231000 | 10.1 |
| | Dry Cake - Dry N2 | 50 | 50 | 0 | 319 | 55600* | 0.2 |
| | Wet Cake | n/a | n/a | n/a | 212 | 238000 | 9.9 |
| | Dry Cake - Humid | 35 | 250 | 60 | 314 | 870* | 10.8 |
| Type 3 | Wet Cake | n/a | n/a | n/a | 141 | 282000 | 6.4 |
| | Dry Cake - Dry N2 | 50 | 50 | 0 | 204 | 33100* | 1.2 |
| | Wet Cake | n/a | n/a | n/a | 287 | 289000 | 6.1 |
| | Dry Cake - Humid | 35 | 250 | 60 | 205 | 70 | 11.5 |

| | | | |
|---|---|---|---|
| specifications | <1000 | <200 | <10 |

The Type 1 solids were observed to convert to Type 2 when washed twice with 3V of 8:1 MeOH:water at room temperature. These solids did not meet specification for residual methanol after drying using dry nitrogen but passed with use of humid nitrogen. Blocky Type 2 solids maintained the Type 2 form upon washing with the methanol solution and did not meet specification for residual methanol for use of dry nitrogen flow. The level of nitrogen improved and was ˜64 times lower with the use of humid nitrogen at 870 ppm. Longer drying times would be needed to remove methanol to lower levels for these solids. Rod/needle-like Type 3 solids also maintain form upon washing. These solids were failing for residual methanol after use of dry nitrogen but passing with drying under humid conditions.

Table VI below provides results for 4 runs using Type 3 sugammadex solids, different wash volumes and age times and shows the results after humid drying for 17 hours at the conditions of 35° C., 250 mmHg, and 60% RH. The solvent composition of the starting wet cake and the final humid dried solids are provided in Table VI.

TABLE VI

| | | 1x Disp. | Age | | Volumes | EtOH | MeOH | Water |
|---|---|---|---|---|---|---|---|---|
| | Sample | Wash 3 V | Time | | Wash | [wt. %] | [ppm] | [wt %] |
| Type 3 | Wet Cake | ✓ | 4 | wk | 5 | 15 | 7590 | 12 |
| Rods | Dry Cake | n/a | n/a | | n/a | 2.1 | ~30 | 8.4 |
| Type 3 | Wet Cake | ✓ | 4 | hr | 3 | 13.4 | 15600 | 8.6 |
| Blocks | Dry Cake | n/a | n/a | | n/a | 4.1 | 534 | 8.0 |
| | Wet Cake | ✓ | 24 | hr | 6 | 17 | 11500 | 11 |
| | Dry Cake | n/a | n/a | | n/a | 2.3 | ~30 | 8.8 |
| | Wet Cake | ✓ | 4 | hr | 8 | 18 | 16300 | 7.9 |

| | | | | | | |
|---|---|---|---|---|---|---|
| Dry Cake | n/a | n/a | n/a | 2.9 | 198 | 8.6 |

| | | | |
|---|---|---|---|
| Specifications (lower limits) | <5 | <200 | <10 |

The initial run was conducted over 4 weeks. After 4 weeks of aging on the benchtop at ambient conditions, these solids were filtered and humid dried to specification as shown in the first set of results above. During the next run, the solids were soaked in 3 volume of wash solution and aged for 4 hours, and the methanol result was at 534 ppm after humid drying. The final two runs used the remaining large Type 3 solids and were conducted using an age time of 24 hours at 6 volumes and 4 hours at 8 volumes respectively. Each of these resulted in dry solids passing specification after humid drying. Additional optimization of the required age time and wash volumes may be possible but aging for 4 hours at 8 volumes appears to be minimum time at the maximum volume needed to achieve successful drying.

The present invention further relates to novel crystalline forms of sugammadex. In particular, the present invention relates to novel crystalline forms of sugammadex designated herein as crystalline form Type 5 of sugammadex and crystalline form Type 11 of sugammadex. The crystalline forms Type 5 and Type 11 of sugammadex described herein may be prepared according to the procedures described below. For Example, crystalline forms Type 5 and Type 11 can be obtained after the humid drying of the wet cake described herein by applying vacuum or a dry nitrogen flow. For each procedure, starting quantities of sugammadex may be obtained from any suitable synthesis, including those described herein and in PCT Publication No. WO2001/040316, Zhang, et al., published Jun. 7, 2001; and WO2019/236436.

### Preparative Example 1: Crystalline Form Type 1 of Sugammadex

Crystalline form Type 1 of sugammadex was prepared as follows:

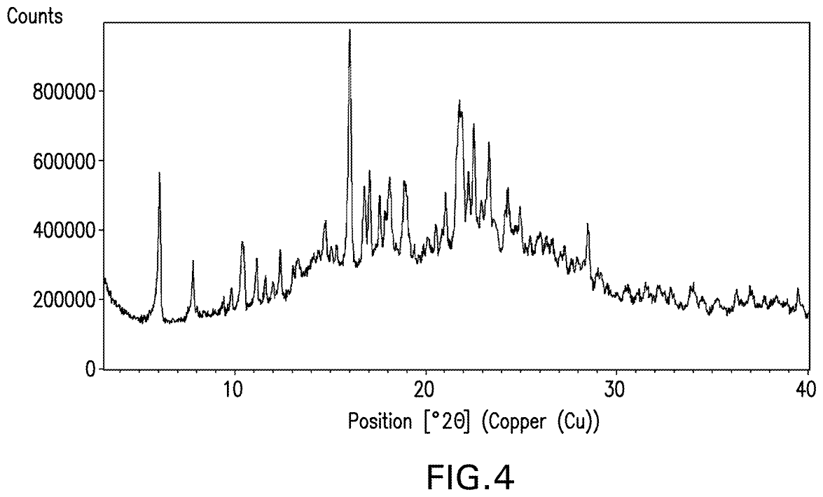

1 g of sugammadex was added to 10 mL of a methanol/water mixture with a 10:1 ratio by volume at 25° C. and while applying magnetic stirring, resulting in a slurry. The slurry was kept at ambient temperature while stirring for 20 hours. A wet cake sample was produced by centrifuging an aliquot of the slurry to a wet paste. PXRD analysis of the wet cake produces the Type 1 pattern. A PXRD pattern of crystalline form Type 1 of sugammadex generated using the equipment and procedures described above is displayed in FIG. 4.

### Preparative Example 2: Crystalline Form Type 2 of Sugammadex

Crystalline form Type 2 of sugammadex was prepared as follows:

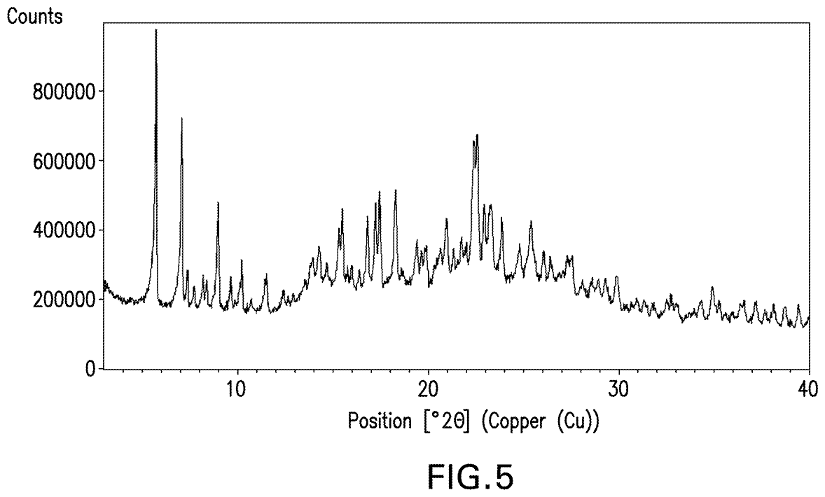

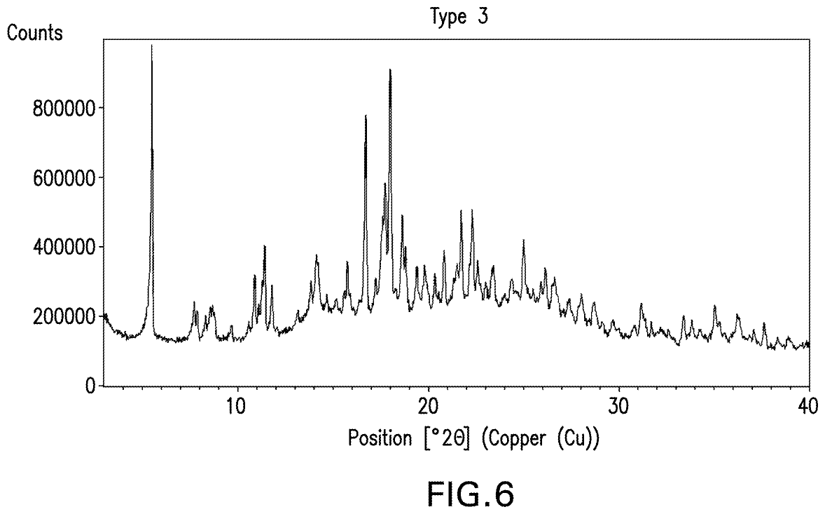

500 mg of sugammadex was added to 5 mL of a methanol/water mixture with a 5/1 ratio by volume at 40° C. and while applying magnetic stirring, resulting in a slurry. The slurry was kept at 40° C. while stirring for 20 hours. A wet cake sample was produced by centrifuging an aliquot of the slurry to a wet paste. PXRD analysis of the wet cake produced the crystalline form Type 2 diffraction pattern substantially as shown in FIG. 5.

### Preparative Example 3: Crystalline Form Type 3 of Sugammadex

Crystalline form Type 3 of sugammadex was prepared as follows:

1 g of sugammadex was added to 10 mL of a methanol/water mixture with a 10/1 ratio by volume at 40° C. while applying magnetic stirring. The resulting slurry was kept at 40° C. while stirring for 3 days. A wet cake sample was produced by centrifuging an aliquot of the slurry to a wet paste. PXRD analysis of the wet cake produced the Type 3 pattern.

### Preparative Example 4: Crystalline Form Type 8 of Sugammadex

Crystalline form Type 8 of sugammadex was prepared as follows:

0.5 g of sugammadex was dissolved in 1.5 mL of water at 25° C. while applying magnetic stirring, resulting in a clear solution. Subsequently, 6 mL of methanol were added over a 5-minute time period while applying slow magnetic stirring, resulting in the precipitation of a solid. The slurry was stirred for another 1 hour at 25° C. A wet cake sample was produced by centrifuging an aliquot of the slurry to a wet paste.

### Preparative Example 5: Crystalline Form Type 9 of Sugammadex

Type 9 appeared as an intermediate and metastable form in a process conducted to generate Type 3. Crystalline form Type 9 of sugammadex was prepared as follows:

A clear solution of 30 g of sugammadex in 90 ml purified water was prepared. The solution was agitated at 200 rpm for 5 min at ambient conditions, heated to 40° C. over 10 minutes, and aged for an additional 10 minutes. Subsequently, several methanol addition and aging steps were conducted as follows: 350 mL of methanol were added linearly over 70 min, producing a slurry. The slurry was aged for 60 minutes, and then 20 ml of methanol was added linearly over 5 minutes followed by the addition of 80 ml of methanol linearly over 30 minutes. The slurry was then aged for 60 minutes until the methanol:water ratio reached 5:1. A wet cake sample was produced by centrifuging an aliquot of the slurry to a wet paste. PXRD analysis of the wet cake produced the Type 9 pattern.

To illustrate the claimed invention Type 1, Type 2, and Type 3 forms of sugammadex solids were prepared as described herein then isolated as a wet cake. To help reduce the humid drying processing time, the wet cake solids may be washed using a standard or aged wash procedure. As would be understood by one of those skilled in the art, standard wash procedures may vary depending on the crystal form and solvent. For example, a standard solvent displacement wash may consist of two washes with 3 volumes of a wash solution of ethanol, methanol, or water, or a mixture thereof. The aged wash may involve soaking the solids in a larger quantity of wash for an extended amount of time. The ratio of ethanol, methanol and water wash can vary depending on the desired specification. Example washes may consist of methanol:water, ethanol:water, ethanol:methanol:water. An example of a standard ethanol wash may consist of ethanol:methanol:water at a ratio, for example, of 86:4:10 vol %. An example of a standard methanol wash may consist of methanol:water at a ratio for example of 3:1 v:v, 5:1 v:v, 8:1 v:v, 9:1 v:v, etc. The number of wash volumes can be decreased or increased based on the estimated capacity of the filter dryer at scale. Wet cake washes are illustrated in Preparative Example 6 and Example 7 below.

### Preparative Example 6: Ethanol:Methanol:Water Slurry of Sugammadex Crystalline Type 2 Form Wet Cake

### Seed Generation:

A seed bed is prepared by charging 758 mg of dry sugammadex solids to a 2.25:1 v:v solution (39 mL) of ethanol and water. The ethanol used is ethanol denatured with 5 v % methanol. The resulting slurry is adjusted to 5° C. in the crystallizer and aged for 30 minutes with agitation.

### Batch Concentrate Preparation:

Crude sugammadex API (3 g) was dissolved in water (9 mL) at room temperature. The pH of the resulting batch concentrate solution was adjusted to 8-9 with NaOH and HCl as needed.

### Seeded Semi-Continuous Crystallization:

Batch concentrate solution (˜11 mL) and denatured (5v % methanol) ethanol (23 mL) were charged simultaneously over ˜3 hours to the prepared seed bed while maintaining 5° C. in the crystallizer. The batch was aged for 30 minutes and ethanol solution (11 mL) was charged over 1 hour resulting in a solvent to water ratio of ˜2.9:1. The batch was aged for 30 minutes at 5° C. and then warmed to 20° C. over 1 hour and aged for another 1 hour at 20° C. The resulting slurry was filtered and displacement washed one time and slurry washed one time with 3 volumes each of an ethanol wash solution (86:4:10 vol % ethanol:methanol:water). The washed wet cake is placed in vacuum oven to dry under vacuum at 50° C. without a nitrogen sweep to afford the dried crystalline product.

### Preparative Example 7: Methanol:Water Slurry of Sugammadex Crystalline Type 2 Form Wet Cake

### Seed Generation:

Humid-dried sugammadex API solids (750 g) were added to a solution of 5:1 v:v MeOH:H₂O (7.5 L). The resulting slurry was aged with agitation at RT for 30 minutes. A slurry sample was removed and the slurry crystals were confirmed to be Form 2 by XRPD and Raman analysis.

### Seeded Crystallization:

Crude sugammadex API (25 kg) was dissolved in H₂O (75 L, 3V) at RT. The solution was heated to 40° C. MeOH (225 L) was added over 1 h. The seed slurry was then added, and the batch was aged at 40° C. for 4 h, followed by a 6 h cooldown to 3° C., and a 9 h age. The batch was filtered and completely deliquored at 3° C., followed by a displacement wash at 20° C. with a solution of 86:4:10 vol % EtOH:MeOH:H₂O (75 L; the EtOH is punctilious). A 4 h soak was then performed, at 20° C. with mild agitation, with a solution of 86:4:10 vol % EtOH:MeOH:H₂O (100 L; the EtOH is punctilious), followed by filtration, to afford the wet cake. PXRD analysis of the wet cake produces the Type 2 pattern.

The following examples illustrate the humid drying of Types 1, 2, 5, 11 and 13.

### Example 1

### Humid Drying Stage: Solvent Removal-Laboratory Process

Additional experiments were completed to fully demonstrate the drying conditions that would be successful for drying of either Type 1 or Type 2 solids and that, once the solvents were removed, water could be successfully reduced to specification as needed. For these runs, the same source of Type 1 or Type 2 wet solids was used and drying was conducted in stages with a 17-hour humid drying stage followed by a 3.5-hour dry nitrogen stage. Conditions for the humid drying stage for each run were as indicated in Table VII. The conditions of the dry nitrogen stage were the same for all runs with each operated at a temperature of 50° C., an outlet pressure of 50 mmHg, and with <1% relative humidity in the drying gas. Residual ethanol, methanol, and water levels for samples taken after the humid drying stage and subsequent dry nitrogen stage were provided for discussion.

TABLE VII
Summary of Process Demonstration Results

| | | Temp | Press | Inlet | EtOH | MeOH | Water |
|---|---|---|---|---|---|---|---|
| Type | Drying Phase | (C.) | (mmHg) | RH % | [wt. %] | [ppm] | [wt %] |
| Type 2 | Humid (17 hrs) | 35 | 250 | 60 | 2.1 | 110 | 8.9 |
| “blocks/plates” | Dry (3.5 hrs) | 50 | 50 | 0 | 2.1 | 100 | 2.0 |
| | Humid (17 hrs) | 35 | 250 | 40 | 3.2 | 390 | 6.5 |
| | Dry (3.5 hrs) | 50 | 50 | 0 | 3.3 | 420 | 1.4 |
| | Humid (17 hrs) | 25 | 250 | 60 | 0.14 | 160 | 19.8 |
| | Dry (3.5 hrs) | 50 | 50 | 0 | 0.15 | 160 | 2.3 |
| | Humid (17 hrs) | 25 | 250 | 40 | 1.8 | 70 | 10.2 |
| | Dry (3.5 hrs) | 50 | 50 | 0 | 2.0 | 80 | 1.6 |
| Type 1 | Humid (17 hrs) | 35 | 250 | 60 | 0.4 | 30 | 9.9 |
| “platelets” | Dry (3.5 hrs) | 50 | 50 | 0 | 0.4 | 20 | 2.0 |
| | Humid (17 hrs) | 35 | 250 | 40 | 1.1 | 30 | 7.4 |
| | Dry (3.5 hrs) | 50 | 50 | 0 | 1.3 | 30 | 1.4 |
| | Humid (17 hrs) | 25 | 250 | 40 | 0.1 | 10 | 13.4 |
| | Dry (3.5 hrs) | 50 | 50 | 0 | 0.1 | 20 | 1.7 |
| | Dry (17 hrs) | 50 | 50 | 0 | 3.4 | 180 | 2.1 |

| | | | |
|---|---|---|---|
| Specifications (lower limits) | <5 | <200 | <10 |

As can be seen, residual ethanol levels were within specification for all cases, consistent with expectations from the earlier probe and DOE experiments. For the humid drying stage, a passing value of 3.3 wt % ethanol was obtained even under conditions where the Type 2 solids were at ˜400 ppm for methanol. A slightly higher residual ethanol value of 3.4 wt % was obtained for the Type 1 run which used dry nitrogen flow only (last row of Table VII). This value was still passing and therefore drying ethanol to specification was not considered an issue for either form.

For methanol, the lowest values for both Types 1 and 2 were obtained for humid conditions of 25° C. and 250 mmHg, and 40% relative humidity. As expected, based on the ease of solvent removal, residual levels of both methanol and ethanol were lower for Type 1 solids as compared with Type 2 solids dried under identical humid drying conditions.

Looking at the last row of Table VII, higher residual solvent levels, although still passing, were obtained when Type 1 solids were dried using dry nitrogen only. Much lower levels were achieved with the use of humid nitrogen flow. This demonstrates the benefits of humidity in the drying gas for solvent displacement by water even the case of the Type 1 form.

At specification lower limits, water levels of ≤10 wt % and solvent levels of methanol, ≤200 ppm and ethanol ≤to about 5 wt % were observed for Types 1, 2 and 3 sugammadex solids under humid conditions at a temperature of about 25° C. to about 35° C., pressure of about 250 mmHg, and about 40% to about 60% relative humidity. Thus, an aspect of the invention is realized when specification levels of residual solvent and water levels are achieved in the humid drying stage of the process. An aspect of this invention is realized when after completion of the humid drying process, the sugammadex solids meet lower limit specifications, wherein water levels are ≤10 wt %, methanol levels are ≤200 ppm and ethanol levels are ≤to about 5 wt %. A subembodiment of this aspect of the invention is realized when humid drying is conducted at a temperature from about 25° C. to about 35° C., a pressure of about 250 mmHg, and about 40% to about 60% relative humidity. Another embodiment of this aspect of the invention is realized when the humid conditions consist of a temperature from about 30° C. to about 35° C., pressure of 250 mmHg, and about 45% to about 60% relative humidity.

### Dry Nitrogen/Vacuum Water Removal Stage

Table VII shows water removal occurred readily during the dry nitrogen stage for all cases and typically resulted in values between 1 and 2 wt %. Residual water levels were more than 1 wt % higher for the Type 1 solids processed under identical humid and dry nitrogen drying stages as the Type 2 and Type 3 solids. Because of the important correlation between residual water levels and methanol removal, this observation possibly explains why solvent removal occurred more readily for Type 1 solids as compared with Type 2 and Type 3. No measurable solvent removal was observed during the dry nitrogen stage for all cases. Slight increase in solvent levels was attributed to the loss of mass due to water removal. Vacuum only drying was also demonstrated to also be equally efficient for water removal (data not shown).

As shown by the data from the laboratory process demonstration and from the DOE model, the residual water content was strongly dependent on the cake drying temperature and to a lesser extent the relative humidity of the drying gas. Therefore as an alternative to the use of dry nitrogen flow or vacuum only drying, the final level of residual water on the solids can be controlled to a target limit (e.g. 6-8 wt %) by either simply adjusting the drying temperature while maintaining the relative humidity level of the humid drying stage or by adjusting the relative humidity while maintaining a constant drying temperature.

### Example 2

### Scaled-Up Process

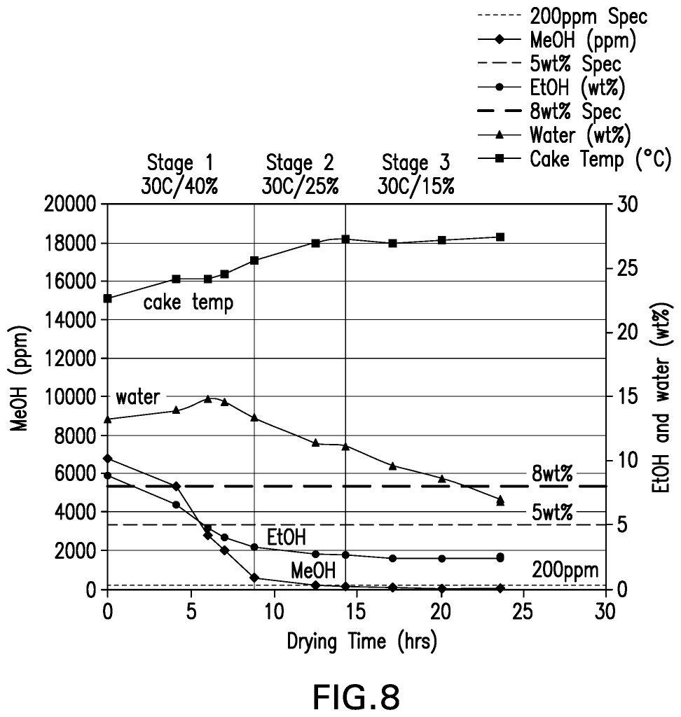

The two-stage humid drying process described above was successfully demonstrated in the pilot plant at the 25 kg scale for several batches and in the manufacturing site at the 250 kg scale. The drying profile was provided in FIG. 8 for a pilot plant batch that was modified slightly from the conditions used during the laboratory confirmation runs. Here, the humid drying stage was conducted at a jacket temperature of 30° C., dryer outlet pressure of 250 mmHg and an inlet humidity of 40%. To slow and limit water removal, two separate water removal conditions were implemented (resulting in an additional stage, Stage 3). Both were conducted at a jacket temperature of 30° C. and dryer outlet pressure of 250 mmHg while the inlet humidity was reduced to 25% during stage 2 and 15% during stage 3.

As can be seen in FIG. 8, the residual solvent levels and cycle times were as expected with drying completed in under 24 hours. Residual ethanol levels were within the desired specification after about 6 hours of processing within the initial humid drying stage. Due to the use of humid conditions, the residual methanol levels continued to drop during stage 2 drying and reached <200 ppm after approximately 12.5 hours. As drying was continued in stage 3, the time to reach the water specification was extended by use of humidity in the drying gas but still reached <10 wt % in approximately 22 hours of total drying time.

### Example 3

### Characterization of Crystalline Form Type 5 of Sugammadex

### Physical Characterization of Type 5 of Sugammadex:

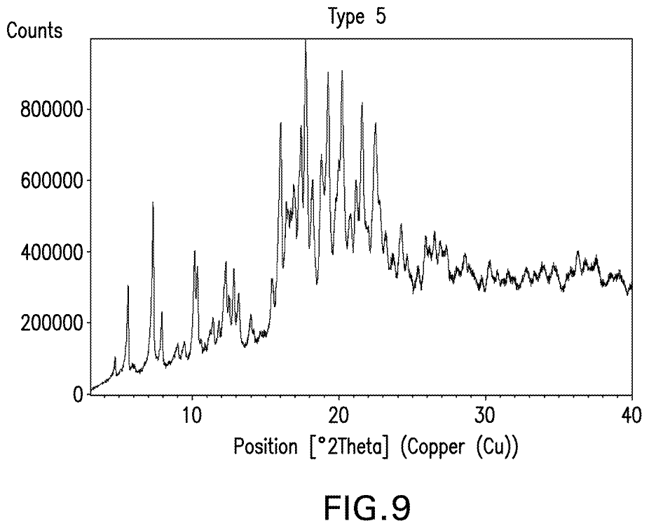

A PXRD pattern of Type 5 of sugammadex generated using the equipment and procedures described above is displayed in FIG. 9.

The intensity of the peaks (y-axis is in counts per second) were plotted versus the 2 theta angle (x-axis is in degrees 2 theta). In addition, the data were plotted with detector counts normalized for the collection time per step versus the 2-theta angle. Peak locations (on the 2-theta x-axis) consistent with these profiles are displayed in Table 1 (+/−0.4° 2 theta). The locations of these PXRD peaks are characteristic for Type 5 of sugammadex. Thus, in another aspect, Type 5 of sugammadex is characterized by a powder x-ray diffraction pattern having each of the peak positions listed in Table VII1, +/−0.4° 2-theta.

TABLE VIII
Diffraction peaks and corresponding
d-spacings for Type 5 of sugammadex

| | Position | d-spacing | Diagnostic |
|---|---|---|---|
| Peak Number | [° Two Theta] | [Å] | Peak Set |
| 1 | 5.58 | 15.80 | 1 |
| 2 | 7.29 | 12.10 | 1 |
| 3 | 10.14 | 8.72 | 1 |
| 4 | 12.26 | 7.21 | 1 |
| 5 | 12.81 | 6.90 | 1 |
| 6 | 16.01 | 5.53 | 1 |
| 7 | 21.56 | 4.12 | 1 |
| 8 | 22.49 | 3.96 | 1 |

In a further aspect, the PXRD peak locations displayed in Table VIII and/or FIG. 9 most characteristic of crystalline form Type 5 of sugammadex can be selected and grouped as “diagnostic peak sets” to conveniently distinguish this crystalline form from others. Selections of such characteristic peaks are set out in Table VIII in the column labeled Diagnostic Peak Set.

Thus, in another aspect, there is provided a crystalline form Type 5 of sugammadex characterized by a powder x-ray diffraction pattern comprising each of the 2-theta values listed in Diagnostic Peak Set 1 in Table VIII, +/−0.2° 2-theta.

### Example 4

### Characterization of Crystalline Form Type 11 of Sugammadex

### Physical Characterization of Type 11 of Sugammadex:

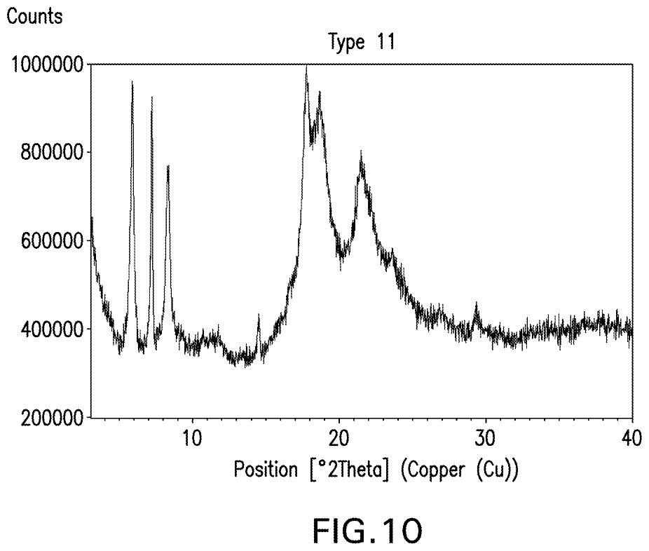

A PXRD pattern of Type 11 of sugammadex generated using the equipment and procedures described above is displayed in FIG. 10.

The intensity of the peaks (y-axis is in counts per second) were plotted versus the 2-theta angle (x-axis is in degrees 2 theta). In addition, the data were plotted with detector counts normalized for the collection time per step versus the 2-theta angle. Peak locations (on the 2-theta x-axis) consistent with these profiles are displayed in Table 2 (+/−0.4° 2 theta). The locations of these PXRD peaks are characteristic for Type 11 of sugammadex. Thus, in another aspect, Type 11 of sugammadex is characterized by a powder x-ray diffraction pattern having each of the peak positions listed in Table IX, +/−0.4° 2-theta.

TABLE IX
Diffraction peaks and corresponding
d-spacings for Type 11 of sugammadex

| | Position | d-spacing | Diagnostic |
|---|---|---|---|
| Peak Number | [° Two Theta] | [Å] | Peak Set |
| 1 | 5.91 | 14.94 | 1 |
| 2 | 7.52 | 12.19 | 1 |
| 3 | 8.38 | 10.53 | 1 |
| 4 | 17.79 | 4.99 | 1 |
| 5 | 18.66 | 4.75 | 1 |
| 6 | 21.52 | 4.15 | 1 |

In a further aspect, the PXRD peak locations displayed in Table IX and/or FIG. 10 most characteristic of crystalline form Type 11 of sugammadex can be selected and grouped as “diagnostic peak sets” to conveniently distinguish this crystalline form from others. Selections of such characteristic peaks are set out in Table IX in the column labeled Diagnostic Peak Set.

Thus, in another aspect, there is provided a crystalline form Type 11 of sugammadex characterized by a powder x-ray diffraction pattern comprising each of the 2-theta values listed in Diagnostic Peak Set 1 in Table IX, +/−0.2° 2-theta.

### Crystalline Form Type 13 of Sugammadex

TABLE X
Diffraction peaks and corresponding d-spacings
for crystalline form Type 13 of sugammadex

| | Position | d-spacing | Diagnostic |
|---|---|---|---|
| Peak Number | [° Two Theta] | [Å] | Peak Set |
| 1 | 6.83 | 12.94 | 2 |
| 2 | 10.12 | 8.74 | 1 |
| 3 | 10.72 | 8.25 | 3 |
| 4 | 11.37 | 7.78 | 3 |
| 5 | 13.90 | 6.37 | 1 |
| 6 | 14.59 | 6.07 | 1 |
| 7 | 16.32 | 5.43 | 1 |
| 8 | 16.73 | 5.30 | 1 |
| 9 | 17.26 | 5.14 | 1 |
| 10 | 18.39 | 4.82 | 1 |
| 11 | 19.42 | 4.57 | 1 |
| 12 | 20.86 | 4.26 | 1 |
| 13 | 21.70 | 4.09 | 3 |
| 14 | 22.12 | 4.02 | 3 |
| 15 | 22.52 | 3.95 | 3 |
| 16 | 22.98 | 3.87 | 3 |
| 17 | 23.34 | 3.80 | 3 |

### Physical Characterization of Type 13 of Sugammadex:

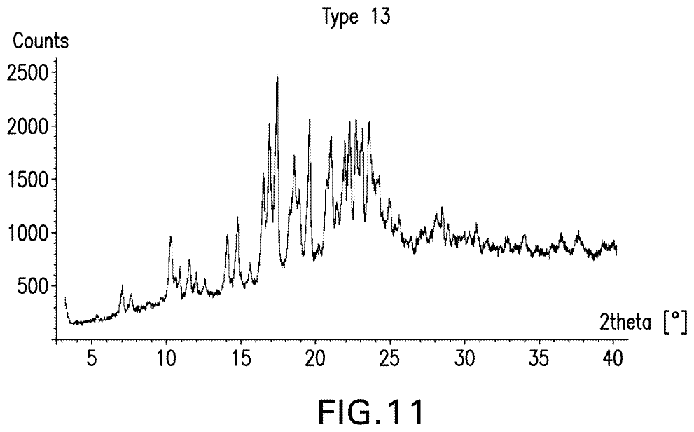

A PXRD pattern of Type 13 of sugammadex generated using the equipment and procedures described above is displayed in FIG. 11.

The intensity of the peaks (y-axis is in counts per second) were plotted versus the 2 theta angle (x-axis is in degrees 2 theta). In addition, the data were plotted with detector counts normalized for the collection time per step versus the 2-theta angle. Peak locations (on the 2-theta x-axis) consistent with these profiles are displayed in Table 1 (+/−0.4° 2 theta). The locations of these PXRD peaks are characteristic for Type 13 of sugammadex. Thus, in another aspect, Type 13 of sugammadex is characterized by a powder x-ray diffraction pattern having each of the peak positions listed in Table VII1, +/−0.4° 2-theta.

In a further aspect, the PXRD peak locations displayed in Table X and/or FIG. 11 most characteristic of crystalline form Type 13 of sugammadex can be selected and grouped as “diagnostic peak sets” to conveniently distinguish this crystalline form from others. Selections of such characteristic peaks are set out in Table X in the column labeled Diagnostic Peak Set.

1. A process for drying crystalline sugammadex wet cake: 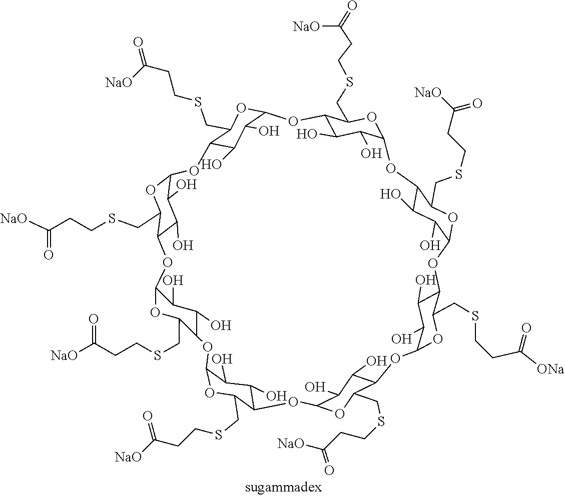
comprising:
1) flowing humid gas through the wet cake of crystalline sugammadex, water, and solvent,
said gas having a relative humidity of about 15% to about 75%,
2) isolating the sugammadex product.

2. The process of claim 1 wherein the humid gas is nitrogen.

3. The process according to claim 1 wherein the solvent is ethanol or methanol, or a mixture thereof.

4. The process according to claim 1 wherein the relative humidity of the nitrogen is about 25% to about 60% and the temperature is maintained from about 25° C. to about 50° C.

5. The process according to claim 4 wherein the relative humidity of the nitrogen is about 25% to about 60% and the temperature is maintained from about 25° C. to about 35° C.

6. The process according to claim 1 wherein the relative humidity of the nitrogen is about 40% to about 45% and the temperature is maintained at about 25° C. to about 50° C.

7. The process according to claim 6 wherein the relative humidity of the nitrogen is about 40% to about 45% and the temperature is maintained from about 25° C. to about 50° C.

8. The process according to claim 1 wherein before isolating the sugammadex product, the residual water level is optionally reduced by applying a dry gas flow or vacuum.

9. The process according to claim 8 wherein the residual water level is reduced by applying a dry nitrogen flow.

10. The process according to claim 8 wherein the residual water level is reduced by vacuum.

11. The process according to claim 9 wherein residual water is reduced using a dry nitrogen flow, or vacuum, at a temperature from about 25° C. to about 50° C. and less than about 10 wt % water.

12. The process according to claim 1 that provides dried crystalline sugammadex with a residual water level of less than or equal to 10 wt %.

13. The process according to claim 1 that provides dried crystalline sugammadex with residual methanol level less than or equal to 200 ppm.

14. The process according to claim 1 that provides dried crystalline sugammadex with residual ethanol level of less than or equal to about 5 wt %.

15. The process according to claim 1 that provides dried crystalline sugammadex with residual water, methanol and ethanol levels of ≤10 wt %, ≤200 ppm, and ≤5 wt %, respectively.

16. The process according to claim 1 wherein pressure during drying process is maintained from 25 mmHg to 475 mmHG.

17. The process according to claim 1 wherein the pressure is 250 mmHg.

18. The process according to claim 1 wherein during drying process residual levels of ≤10 wt % water, ≤200 ppm methanol, and ≤5 wt % ethanol are achieved in a single step.

19. The process according to claim 1 wherein residual levels of ≤10 wt % water, ≤200 ppm methanol, and ≤5 wt % ethanol are achieved by flowing humid nitrogen with a relative humidity of about 40% to about 45% and at a temperature of about 30° C. to about 35° C. through the wet cake of crystalline sugammadex, water, and solvent, then drying with a dry nitrogen flow or vacuum.

20. A crystalline form Type 5 of sugammadex characterized by a powder x-ray diffraction pattern with at least peaks at diffraction angles degrees 2 theta (+/−0.2°) of:
5.58
7.29
10.14
12.26
12.81
16.01
21.56
22.49

21. A crystalline form Type 11 of sugammadex characterized by a powder x-ray diffraction pattern with at least peaks at diffraction angles degrees 2 theta (+/−0.2°) of:
5.91
7.52
8.38
17.79
18.66
21.52
